(19) United States
(12) Patent Application Publication
(10) Pub. No.: US 2024/0044180 A1
(43) Pub. Date: Feb. 8, 2024

(54) MEDICAL NONMAGNETIC RESTRAINT ASSEMBLY AND METHOD

(71) Applicant: Darryl Foy, Joliet, IL (US)

(72) Inventor: Darryl Foy, Joliet, IL (US)

(21) Appl. No.: 17/880,813

(22) Filed: Aug. 4, 2022

(51) Int. Cl. E05B 75/00; G01R 33/28

(52) U.S. Cl. CPC E05B 75/00; G01R 33/288; A61B 5/055

(57) ABSTRACT

A medical nonmagnetic restraint assembly and method for being compatible with a magnetic resonance imaging machine includes a hand restraint made of a plastic material. The plastic material is nonmagnetic thus being compatible with a magnetic resonance imagine machine. The hand restraint has a pair of wrist cuffs, and each of the wrist cuffs has a locking mechanism. An ankle restraint made of the plastic material has a pair of ankle cuffs. Each of the ankle cuffs has a locking mechanism as well. Additionally, a leg restraint made of the plastic material restraint has a pair of leg cuffs. Each of the leg cuffs has a strap engaging with a locking mechanism of the leg cuff.

### (b) CROSS-REFERENCE TO RELATED APPLICATIONS

Not Applicable

### (c) STATEMENT REGARDING FEDERALLY SPONSORED RESEARCH OR DEVELOPMENT

Not Applicable

### (d) THE NAMES OF THE PARTIES TO A JOINT RESEARCH AGREEMENT

Not Applicable

### (e) INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### (f) STATEMENT REGARDING PRIOR DISCLOSURES BY THE INVENTOR OR JOINT INVENTOR

Not Applicable

### (g) BACKGROUND OF THE INVENTION

### (1) Field of the Invention

The disclosure relates to restraint device and more particularly pertains to a new restraint device for being compatible with a magnetic resonance imaging machine.

### (2) Description of Related Art Including Information Disclosed Under 37 CFR 1.97 and 1.98

The prior art relates to restraint devices. The prior art relates to a variety of restraint devices being made of a plastic material. Known prior art lacks a restraint device made of a plastic material configured for restraining a pair of wrists, a pair of ankles, and a pair of calves of the user while engaging with a magnetic resonance imaging machine.

### (h) BRIEF SUMMARY OF THE INVENTION

An embodiment of the disclosure meets the needs presented above by generally comprising a hand restraint made of a plastic material. The plastic material is configured for being nonmagnetic wherein being compatible with a magnetic resonance imagine machine. The hand restraint has a pair of wrist cuffs, wherein each of the wrist cuffs has a locking mechanism. An ankle restraint made of the plastic material has a pair of ankle cuffs. Each of the ankle cuffs has a locking mechanism as well. Additionally, a leg restraint made of the plastic material restraint has a pair of leg cuffs. Each of the leg cuffs has a strap being configured for engaging with a locking mechanism of the leg cuff.

Another embodiment of the disclosure includes a method comprising the step of fastening a pair of wrist cuffs of a hand restraint around a pair of wrists of the user prior to securing a latch of each of the wrist cuffs in a closed position with a locking mechanism. Additionally fastening a pair of ankle cuffs of an ankle restraint around a pair of ankles of the user and securing a latch of each of the ankle cuffs in a closed position with a locking mechanism. Furthermore, fastening a pair of leg cuffs of a leg restraint around a pair of calves of the user prior to securing a strap of each of the leg cuffs in a closed position with a locking mechanism. Subsequently, engaging the user with a magnetic resonance imaging machine wherein the nonmagnetic restraint assembly being compatible with the magnetic resonance imagining machine.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### (i) BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a front view of a medical nonmagnetic restraint assembly and method according to an embodiment of the disclosure.

FIG. 2 is an isometric detail view of an embodiment of the disclosure.

FIG. 3 is an in-use view of an embodiment of the disclosure.

### (j) DETAILED DESCRIPTION OF THE INVENTION

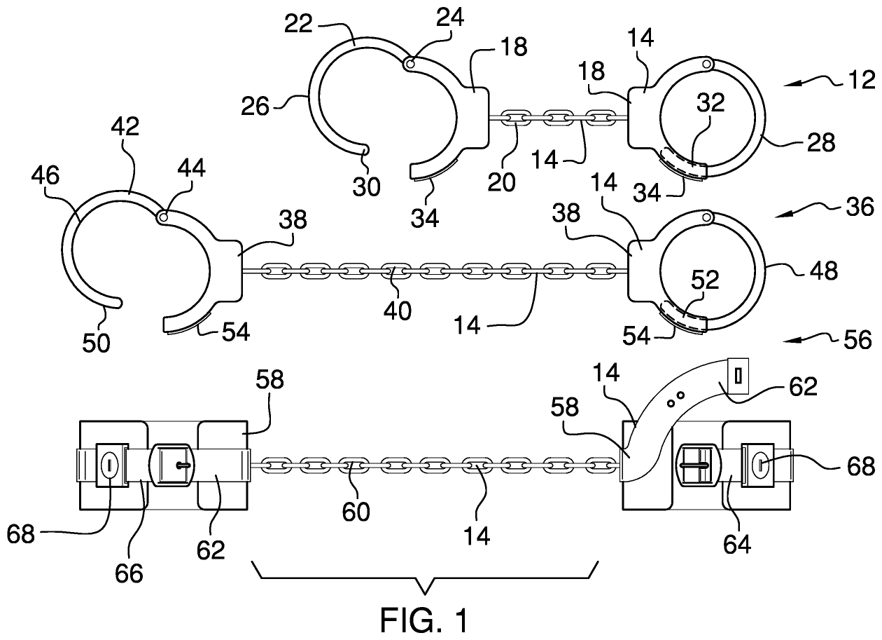

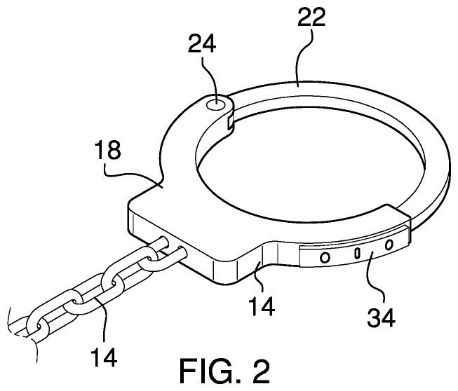

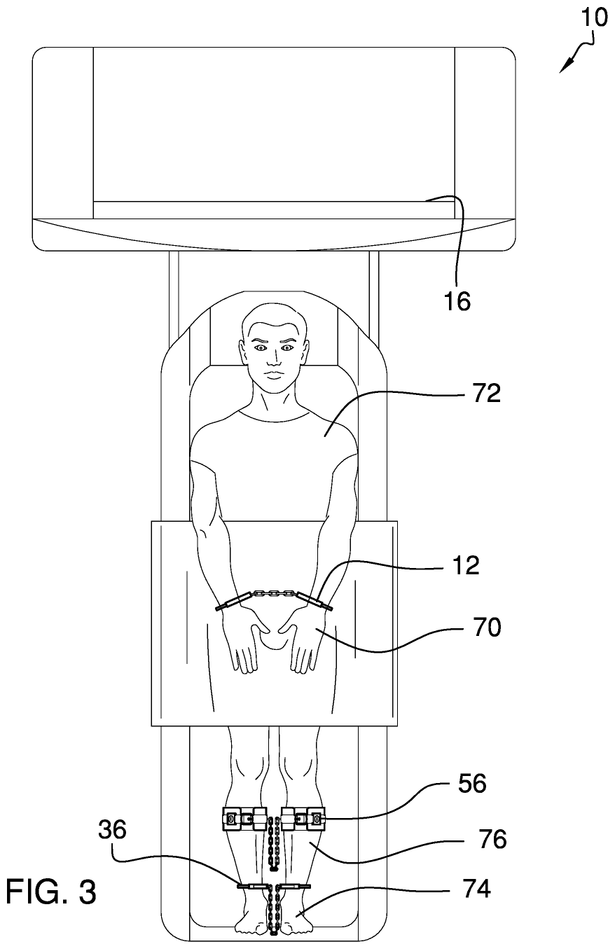

With reference now to the drawings, and in particular to FIGS. 1 through 3 thereof, a new restraint device embodying the principles and concepts of an embodiment of the disclosure and generally designated by the reference numeral 10 will be described.

As best illustrated in FIGS. 1 through 3, the medical nonmagnetic restraint assembly and method 10 generally comprises a hand restraint 12 made of a plastic material 14. The plastic material 14 is configured for being nonmagnetic wherein being compatible with a magnetic resonance imagine machine 16. The hand restraint 12 has a pair of wrist cuffs 18 being coupled to each other by a chain 20. The chain 20 of the hand restraint 12 is made of the plastic material 14. Each of the wrist cuffs 18 has a latch 22 having a hinge 24 being configured for positioning the latch 22 of each of the wrist cuffs 18 from an open position 26 to a closed position 28 and vise versa. The closed position 28 of the latch 22 of each of the wrist cuffs 18 is configured for nesting an end 30 of the latch 22 within a cavity 32 of the wrist cuff 18. Each of the wrist cuffs 18 has a locking mechanism 34 being configured for retaining the latch 22 in a closed position 28 relative to the wrist cuff 18. The locking mechanism 34 has a key being configured for engaging with the locking mechanism 34.

An ankle restraint 36 made of the plastic material 14 has a pair of ankle cuffs 38 coupled to each other by a chain 40. The chain 40 of the ankle restraint 36 is made of the plastic material 14. Each of the ankle cuffs 38 has a latch 42 having a hinge 44 being configured for positioning the latch 42 of each of the ankle cuffs 38 from an open position 46 to a closed position 48 and vise versa. The closed position 48 of the latch 42 of each of the ankle cuffs 38 is configured for nesting an end 50 of the latch 42 within a cavity 52 of the ankle cuff 38. Each of the ankle cuffs 38 has a locking mechanism 54 being configured for retaining the latch 42 in a closed position 48 relative to the ankle cuff 38.

A leg restraint 56 made of the plastic material 14 has a pair of leg cuffs 58 coupled to each other by a chain 60. The chain 60 of the leg restraint 56 is made of the plastic material 14. Each of the leg cuffs 58 has a strap 62 being configured for positioning from an open position 64 to a closed position 66 and vise versa. The strap 62 is configured for engaging with a locking mechanism 68 of each of the leg cuffs 58. The locking mechanism 68 of each of the leg cuffs 58 is configured for retaining the strap 62 in the closed position 66 relative to the leg cuff 58.

In use, the pair of wrist cuffs 18 of the hand restraint 12 fastens around a pair of wrists 70 of a user 72 prior to securing the latch 22 of each of the wrist cuffs 18 in the closed position 28 with the locking mechanism 34. Additionally, the pair of ankle cuffs 38 of the ankle restraint 36 fastens around a pair of ankles 74 of the user 72 before securing the latch 42 of each of the ankle cuffs 38 in the closed position 48 with the locking mechanism 54. Furthermore, the pair of leg cuffs 58 of the leg restraint 56 fastens around a pair of calves 76 of the user 72 prior to securing the strap 62 of each of the leg cuffs 58 in the closed position 66 with the locking mechanism 68. Subsequently, the user 72 engages with the magnetic resonance imaging machine 16 wherein the nonmagnetic restraint assembly 10 is compatible with the magnetic resonance imagining machine 16. The medical nonmagnetic restraint assembly and method 10 is configured for use on prisoners who need to seek medical attention using the magnetic resonance imagining machine 16.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

1. A nonmagnetic restraint assembly configured for being compatible with a magnetic resonance imaging machine, the nonmagnetic restraint assembly comprising:
a hand restraint made of a plastic material, the plastic material being configured for being nonmagnetic wherein being compatible with a magnetic resonance imagine machine, the hand restraint having a pair of wrist cuffs, each of the wrist cuffs having a locking mechanism;
an ankle restraint made of the plastic material, the ankle restraint having a pair of ankle cuffs, each of the ankle cuffs having a locking mechanism; and
a leg restraint made of the plastic material, the leg restraint having a pair of leg cuffs, each of the leg cuffs having a strap coupled to and extending around a band, the strap being centered between and parallel to opposed longitudinal edges of the band, the strap being configured for engaging with a locking mechanism of the leg cuff.

2. The nonmagnetic restraint assembly of claim 1, further comprising the pair of wrist cuffs being coupled to each other by a chain, said chain of said hand restraint being said plastic material.

3. The nonmagnetic restraint assembly of claim 1, further comprising each of the wrist cuffs having a latch, the latch of each of the wrist cuffs having a hinge being configured for positioning the latch of each of the wrist cuffs from an open position to a closed position and vise versa, the closed position of the latch of each of the wrist cuffs being configured for nesting an end of the latch within a cavity of the wrist cuff.

4. The nonmagnetic restraint assembly of claim 3, further comprising the locking mechanism being configured for retaining the latch in a closed position relative to the wrist cuff.

5. (canceled)

6. The nonmagnetic restraint assembly of claim 1, further comprising the pair of ankle cuffs being coupled to each other by a chain, said chain of said ankle restraint being said plastic material.

7. The nonmagnetic restraint assembly of claim 4, further comprising each of the ankle cuffs having a latch, the latch of each of the ankle cuffs having a hinge being configured for positioning the latch of each of the ankle cuffs from an open position to a closed position and vise versa, the closed position of the latch of each of the ankle cuffs being configured for nesting an end of the latch within a cavity of the ankle cuff.

8. The nonmagnetic restraint assembly of claim 7, further comprising the locking mechanism being configured for retaining the latch in a closed position relative to the ankle cuff.

9. The nonmagnetic restraint assembly of claim 1, further comprising the pair of leg cuffs being coupled to each other by a chain, said chain of said leg restraint being said plastic material.

10. The nonmagnetic restraint assembly of claim 8, further comprising the strap of each of the leg cuffs being configured for positioning from an open position to a closed position and vise versa, the strap being configured for engaging with a locking mechanism of each of the leg cuffs, the locking mechanism of each of the leg cuffs being configured for retaining the strap in the closed position relative to the leg cuff.

11. A nonmagnetic restraint assembly configured for being compatible with a magnetic resonance imaging machine, the nonmagnetic restraint assembly comprising:
a hand restraint made of a plastic material, the plastic material being configured for being nonmagnetic wherein being compatible with a magnetic resonance imagine machine, the hand restraint having a pair of wrist cuffs, the pair of wrist cuffs being coupled to each other by a chain, said chain of said hand restraint being said plastic material, each of the wrist cuffs having a latch, the latch of each of the wrist cuffs having a hinge being configured for positioning the latch of each of the wrist cuffs from an open position to a closed position and vise versa, the closed position of the latch of each of the wrist cuffs being configured for nesting an end of the latch within a cavity of the wrist cuff, each of the wrist cuffs having a locking mechanism, the locking mechanism being configured for retaining the latch in a closed position relative to the wrist cuff;
an ankle restraint made of the plastic material, the ankle restraint having a pair of ankle cuffs, the pair of ankle cuffs being coupled to each other by a chain, said chain of said ankle restraint being said plastic material, each of the ankle cuffs having a latch, the latch of each of the ankle cuffs having a hinge being configured for positioning the latch of each of the ankle cuffs from an open position to a closed position and vise versa, the closed position of the latch of each of the ankle cuffs being configured for nesting an end of the latch within a cavity of the ankle cuff, each of the ankle cuffs having a locking mechanism, the locking mechanism being configured for retaining the latch in a closed position relative to the ankle cuff; and
a leg restraint made of the plastic material, the leg restraint having a pair of leg cuffs, the pair of leg cuffs being coupled to each other by a chain, said chain of said leg restraint being said plastic material, each of the leg cuffs having a strap coupled to and extending around a band, the strap being centered between and parallel to opposed longitudinal edges of the band, the strap of each of the leg cuffs being configured for positioning from an open position to a closed position and vise versa, the strap being configured for engaging with a locking mechanism of each of the leg cuffs, the locking mechanism of each of the leg cuffs being configured for retaining the strap in the closed position relative to the leg cuff.

12. (canceled)
